(19) United States
(12) Patent Application Publication
(10) Pub. No.: US 2017/0348542 A1
(43) Pub. Date: Dec. 7, 2017

(54) SYSTEM FOR TREATMENT BY PHOTODYNAMIC THERAPY AND METHOD FOR PREPARATION OF SUCH SYSTEM

(71) Applicants: INSERM (INSTITUT NATIONAL DE LA SANTE ET DE LA RECHERCHE MÉDICALE), Paris (FR); CENTRE HOSPITALIER REGIONAL ET UNIVERSITAIRE DE LILLE (CHRU), Lille (FR); UNIVERSITE DE LILLE 2 DROIT ET SANTE, Lille (FR)

(72) Inventors: Nacim BETROUNI, Lille (FR); Serge MORDON, Lille (FR); Camille MUNCK, Loos (FR); Jean-Claude LESAGE, Loos (FR); Arnaud SCHEREREEL, Loos (FR)

(21) Appl. No.: 15/544,143

(22) PCT Filed: Jan. 19, 2016
(86) PCT No.: PCT/EP2016/050968

§ 371 Date: Jul. 17, 2017

(30) Foreign Application Priority Data
Jan. 19, 2015 (EP) 15305052.1

(51) Int. Cl. A61N 5/06; A61B 18/22; A61B 90/30; A61N 5/067; A61B 34/20; A61B 90/00

(52) U.S. Cl. CPC A61N 5/0603; A61N 5/062; A61N 2005/063; A61B 2034/2051; A61B 2090/373; A61B 2090/3612; A61B 2090/306; A61N 2005/067; A61B 2018/2261

(57) ABSTRACT

System for treatment by photodynamic therapy comprising: —an illuminating device (10) including a light emitting surface for illuminating an internal surface to be treated with a light adapted to activate a photosensitizer compound, the light emitting surface emitting light with a distribution of light power comprising fractions of light power decreasing from a maximum at the light emitting surface, the light emitting surface having a determined illumination profile that provides respective illuminated areas for a plurality of the fractions of light power, —a positioning system (40) adapted to position in real-time the light emitting surface within a reference frame, —an electronic unit (45) connected to the positioning system (40) and adapted to monitor in real-time a dose of light energy delivered to the internal surface based on the illumination profile and the position of the light emitting surface.

The invention relates to a system for treatment by photodynamic therapy and to a method for preparation of such system.

In particular, the invention relates to a system for treatment by photodynamic therapy of an internal surface of a patient's body, the internal surface being delimited by tissues comprising cells having a photosensitizer compound absorbed therein.

Although not limited thereto, the invention finds particular applications to the treatment of cancerous tumours of the pleural cavity, such as malignant pleural mesothelioma (MPM) or peritoneal carcinoma.

The malignant pleural mesothelioma is a tumour of the pleura of which the main etiologic factor is an anterior exposition, most of time professional, to asbestos fibers. The malignant pleural mesothelioma is often diagnosed 30 to 40 years after exposition. It can more rarely concern other serosa such as peritoneum, pericardia and exceptionally the vaginal coat of testis. In France, the current occurrence of the malignant pleural mesothelioma is estimated to 900 cases per year. Considered as a rare cancer, its occurrence is however increasing across the world since 1960 with a peak expected in Europe during the next decades. This occurrence could become important in emerging countries still using asbestos nowadays, especially for its insulating properties.

Regarding surgical treatment, two types of intervention are currently implemented. First, the extra-pleural pneumonectomy (PEP), which is a block pleural pulmonary resection enlarged to pericardia and diaphragm, is only envisioned at a limited stage of the cancer and for patients carefully selected since it implies high intraoperative mortality (5%) and morbidity (>50%), as well as a risk of alteration of life quality. Second, the pleurectomy/decortication (P/D), exeresis of the parietal pleura and decortication of the visceral pleura, less disabling, can be envisioned for more patients but does not imply a satisfying resection in a carcinological point of view and the risk of locoregional recurrence is high.

Nevertheless, no curative surgical treatment has been validated to date and a multimodal treatment, for example associated with chemotherapy and/or radiotherapy, is usually implemented to eradicate microscopic tumour cells.

In this context of multimodal treatment, photodynamic therapy (PDT) has been taken into consideration for association with surgery. The photodynamic therapy rests the on interaction of three components: a photosensitizer compound, oxygen within the tissues and light having properties suitable for activating the photosensitizer compound. The photosensitizer compound injected within the body of the patient is absorbed by all cells but remains a longer time within the tumour cells. Upon activation of the photosensitizer compound by the light, photo-chemical reactions occur resulting in a destruction of the tumour cells.

Regarding the pleural cavity, the treatment may comprise the two following steps:

injection of the photosensitizer compound a predetermined time period, for example of at least 24 hours, prior to surgery,

intra-operative illumination of the pleural cavity after maximal tumour resection surgery, for example by laser.

Pleural cavity is a space geometrically complex to illuminate, especially due to diaphragmatic dead-ends, mediastina folds and the presence of organs such as the heart, the oesophagus, the lung and the vessels.

A system for treatment by photodynamic therapy of the pleural cavity has been disclosed by DeLaney et al. in “A light-diffusion device for intraoperative photodynamic therapy in the peritoneal or pleural cavity”, Journal of clinical laser medicine & surgery—October 1991, 361-366. The system for treatment comprises an illuminating device including a light emitting surface for illuminating an internal surface to be treated with a light adapted to activate a photosensitizer compound, and an electronic unit adapted to monitor in real-time a dose of light energy delivered to the internal surface. In particular, the electronic unit comprises a set of isotropic sensors attached to the internal cavity to be treated, and a Wattmeter adapted to measure light power at each isotropic sensor. DeLaney et al. discloses a known distribution of light power at a periphery of a balloon arranged at a distal end of the illuminating device.

In a similar manner, WO 2013/144830 discloses a system for treatment by photo therapy comprising an illuminating device provided with a plurality of discrete light emitting surfaces. Light is emitted with a known distribution of light power at the light emitting surfaces.

However, the efficiency of photodynamic therapy is closely linked to the illumination of the surfaces of the cavity. The know systems for treatment, which only provide light power at one sole determined level (that of the balloon or that of the light emitting surface) or doses of light energy delivered on the surfaces of the isotropic sensors, do not provide a sufficient knowledge of the distribution of light power to enable an homogenous and complete illumination of the internal surface to be ensured.

The invention aims to solve the above mentioned problems.

To this end, according to a first aspect, the invention provides a system for treatment by photodynamic therapy comprising:

an illuminating device intended for illuminating the internal surface to be treated, the illuminating device including a light emitting surface for emitting a light adapted to activate the photosensitizer compound, the illuminating device being adapted to diffuse the light emitted by the light emitting surface with a distribution of light power comprising fractions of light power decreasing from a maximum at the light emitting surface,

an electronic unit adapted to monitor in real-time a dose of light energy delivered to the internal surface,

wherein the system for treatment further comprises a positioning system adapted to position in real-time the light emitting surface within a reference frame, and wherein the light diffused by the light emitting surface has a determined illumination profile that provides respective illuminated areas for a plurality of the fractions of light power so that the dose of light energy delivered at each distance from the light emitting surface of the plurality of the fractions of light power is known, the electronic unit being connected to the positioning system and adapted to monitor in real-time the dose of light energy based on the illumination profile and the position of the light emitting surface.

Hence, the invention proposes an improved system for treatment by photodynamic therapy based on the combination of a theoretical illumination profile of the illuminating device with a spatial positioning system enabling movements of the illuminating device within the cavity to be monitored in real-time. The whole distribution of diffused light is characterized so that the dose of light energy delivered at each distance from the light emitting surface of the plurality of the fractions of light power is known. With such characterization, any doses of light energy delivered to each portion of the surface to be treated can be taken into account, including doses of light energy delivered when the illuminating device is moved within the cavity or when adjacent portions are illuminated. The treatment can then be controlled so that the cavity to be treated can be subjected to homogenous and complete illumination associated with an optimal and accurate dosimetry, namely a light dose per surface unit.

Moreover, the system for treatment according to the invention offers an easier and less invasive implementation than that of the known system which requires sensors to be arranged within the cavity to be treated.

According to some provisions, the illuminating device can be standardized thereby rendering the optimal illumination easily repeatable.

Also, the system for treatment allows a visual representation of the light dose distributed over the surface of the cavity displayed on images during the surgical intervention. The system for treatment can then be made easier and more intuitive to use.

The positioning system may comprise at least one positioning sensor attached to the illuminating device and connected to the electronic unit, the positioning sensor being adapted to emit a signal representative of the position of said positioning sensor within the reference frame.

In particular, the positioning system may be an electromagnetic spatial tracking system comprising an electromagnetic sensor as positioning sensor, and a transmitter adapted to generate an electromagnetic field.

The electronic unit may be provided with an image of the internal surface to be treated and may comprise a displaying device adapted to display the image of the internal surface to be treated, the electronic unit being adapted to display in real-time an image of the light emitting surface on the image of the internal surface to be treated.

The electronic unit may be further adapted to display in real-time the dose of light energy delivered to the internal surface to be treated.

The displaying device may comprise an image parameter representative of the dose of light energy on the image of the internal surface to be treated, the image parameter comprising a plurality of values each representative of a value of the dose of light energy, the electronic unit being adapted to change in real-time the value of the image parameter in accordance with the value of the dose of light energy.

The electronic unit may be further adapted to stop illumination of the internal surface to be treated when a determined threshold of illumination has been reached.

The illuminating device may comprise an illuminating member extending along a central axis between opposed proximal and distal ends, the light emitting surface being arranged at the distal end and extending along the central axis so as to emit the light transversely with respect to the central axis.

The illuminating member may comprise:

a sheath having an enlarged portion arranged at the distal end, centered on the central axis and filed with a light diffusing solution, and

a core extending in the sheath and carrying the light emitting surface arranged within the enlarged portion.

The core of the illuminating member may be an optical fiber having a proximal end and a distal end which carries the light emitting surface, and the illuminating device may further comprise a laser light source connected to the proximal end of the optical fiber.

The distribution of light power emitted by the light emitting surface may have a rotational symmetry, the illuminated areas for the plurality of the fractions of light power being centered on the light emitting surface.

The light emitting surface may be unique and cylindrical along the central axis.

According to a second aspect, the invention provides a method for preparation of a system for treatment by photodynamic therapy of an internal surface of a patient's body, the internal surface being delimited by tissues comprising cells having a photosensitizer compound absorbed therein, the method for preparation comprising the steps of:

providing an illuminating device intended for illuminating the internal surface to be treated, the illuminating device including a light emitting surface for emitting with a light adapted to activate the photosensitizer compound, the illuminating device being adapted to diffuse light emitted by the light emitting surface with a distribution of light power comprising fractions of light power decreasing from a maximum at the light emitting surface,

determining an illumination profile of the light diffused by the light emitting surface, the illumination profile providing respective illuminated areas for a plurality of the fractions of light power,

providing a positioning system adapted to position in real-time the light emitting surface within a reference frame,

providing an electronic unit connected to the positioning system and adapted to monitor in real-time a delivered dose of light energy on the illumination profile and the position of the light emitting surface.

The step of determining the illumination profile may comprise:

measuring an efficient attenuation coefficient μ_{eff} of light,

calculating the fractions of light power based on the Beer-Lambert equation: I=I₀.exp(−μ_{eff}.z) wherein I is the intensity of light, I₀ is the incident intensity of light and z is the distance to the light emitting surface,

measuring the respective illuminated areas for the plurality of the fractions of light power.

Measuring the efficient attenuation coefficient may comprise measuring light power at a plurality of distances from the light emitting surface, ploting an interpolation curve y=a.exp(−bx) representing light power as a function of a distance to the light emitting surface, and assigning b to the efficient attenuation coefficient μ_{eff} in accordance with the Beer-Lambert equation.

Measuring the respective illuminated areas for the plurality of the fractions of light power may comprise taking a picture illustrating the distribution of light power, identifying the plurality of the fractions of light power and delimiting respective borders of the plurality of the fractions of light power, calculating an area of each border corresponding to one of the illuminated areas.

The method for preparation may further comprise the steps of:

acquiring an image of the internal surface to be treated,

providing the electronic unit with the image of the internal surface to be treated,

displaying the image of the internal surface to be treated on a displaying device of the electronic unit.

These provisions enable an image of the light emitting surface to be displayed in real-time on the image of the internal surface to be treated, and possibly enable the dose of light energy delivered to the internal surface to be treated to be displayed in real-time, in particular by changing in real-time a value of an image parameter representative of the dose of light energy on the image of the internal surface to be treated in accordance with a value of the dose of light energy.

The method for preparation may further comprise the step of arranging the image of the internal surface to be treated in the reference frame of the positioning system.

Other objects and advantages of the invention will emerge from the following disclosure of a particular embodiment of the invention given as non limitative example, the disclosure being made in reference to the enclosed drawings in which:

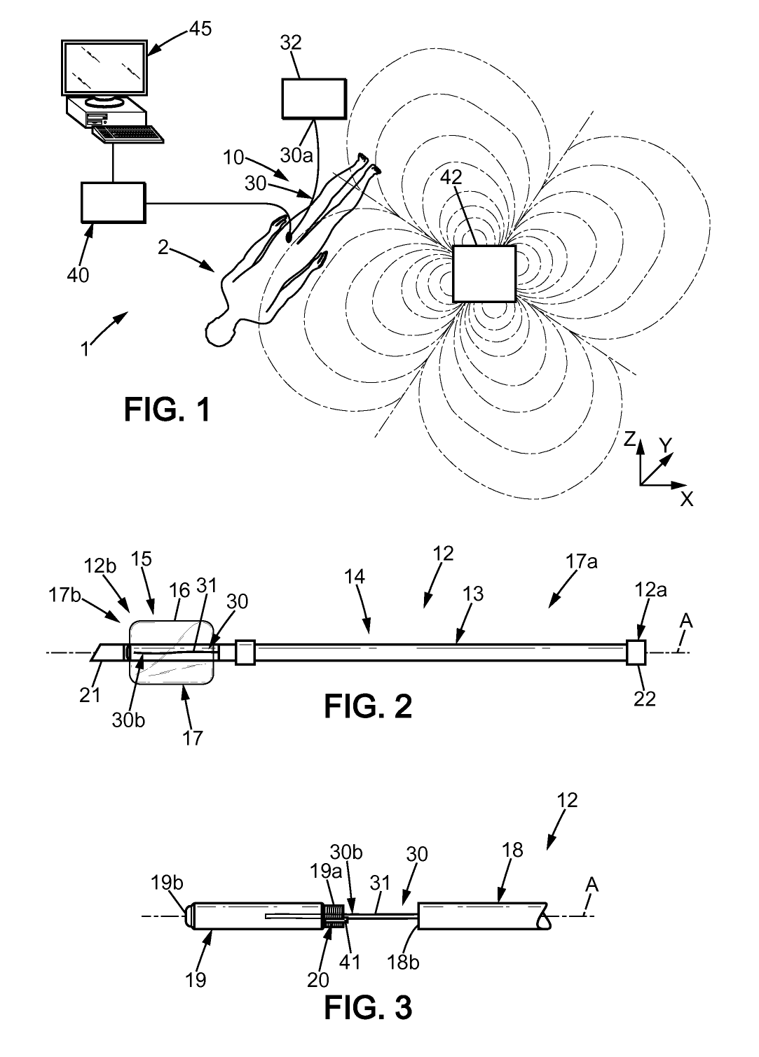

FIG. 1 is a schematic representation of a system for treatment by photodynamic therapy of an internal surface of a patient's body according to an embodiment of the invention, the system for treatment comprising an illuminating device having a light emitting surface, a positioning system adapted to position in real-time the light emitting surface and an electronic unit,

FIG. 2 is a representation of an illuminating member of the illuminating device of the system for treatment of FIG. 1,

FIG. 3 is a partial representation of a distal end of the illuminating member of FIG. 2, illustrating an optical fiber as a core having the light emitting surface arranged within a tube and an end cap of a sheath,

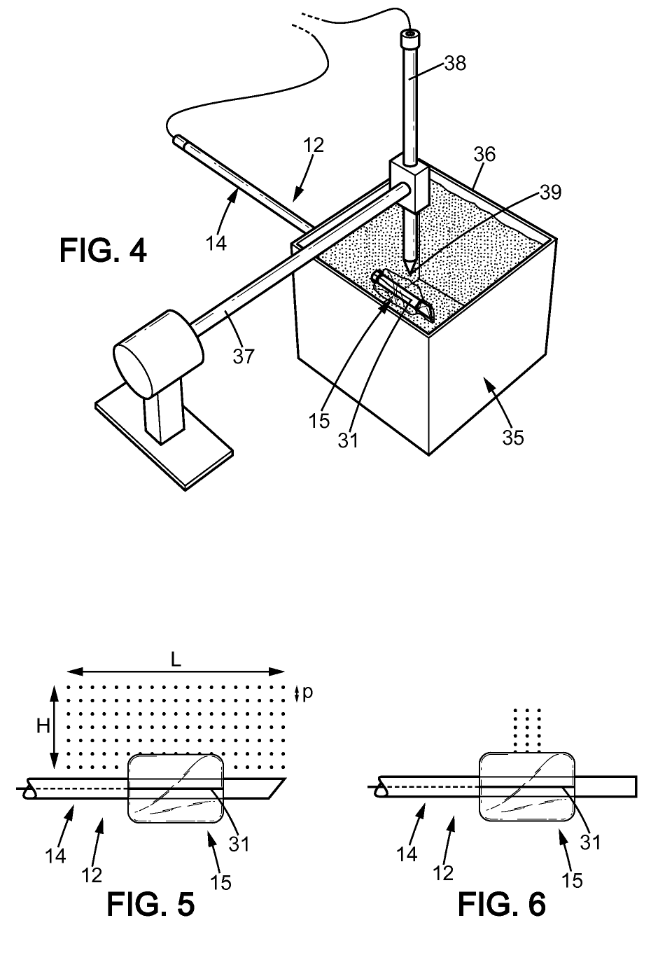

FIG. 4 is a schematic representation of a test bench implemented to determine an illumination profile of the light emitting surface of the illuminating member of FIG. 2, the illumination profile providing respective illuminated areas for a plurality of fractions of light power, the light emitting surface being placed within a tank filed with a light diffusing solution, and an isotropic sensor connected to a Wattmeter measuring light power of the light emitting surface of the illuminating member,

FIGS. 5 and 6 are schematic representation of measurements of light power performed with the test bench of FIG. 4,

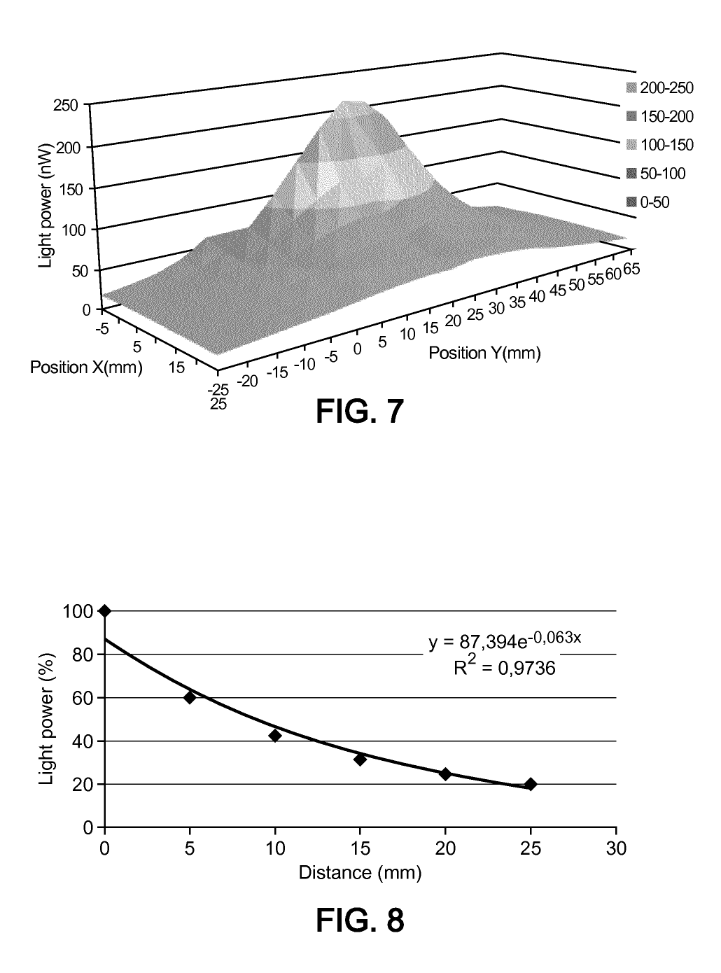

FIG. 7 is a graph illustrating the light power as a function of the position of the isotropic sensor resulting from the measurements of FIG. 5,

FIG. 8 is a graph illustrating the percentage of light power as a function of the distance to the light emitting surface resulting from the measurements of FIG. 5, and an interpolation exponential curve providing an efficient attenuation coefficient of the light in the diffusing solution,

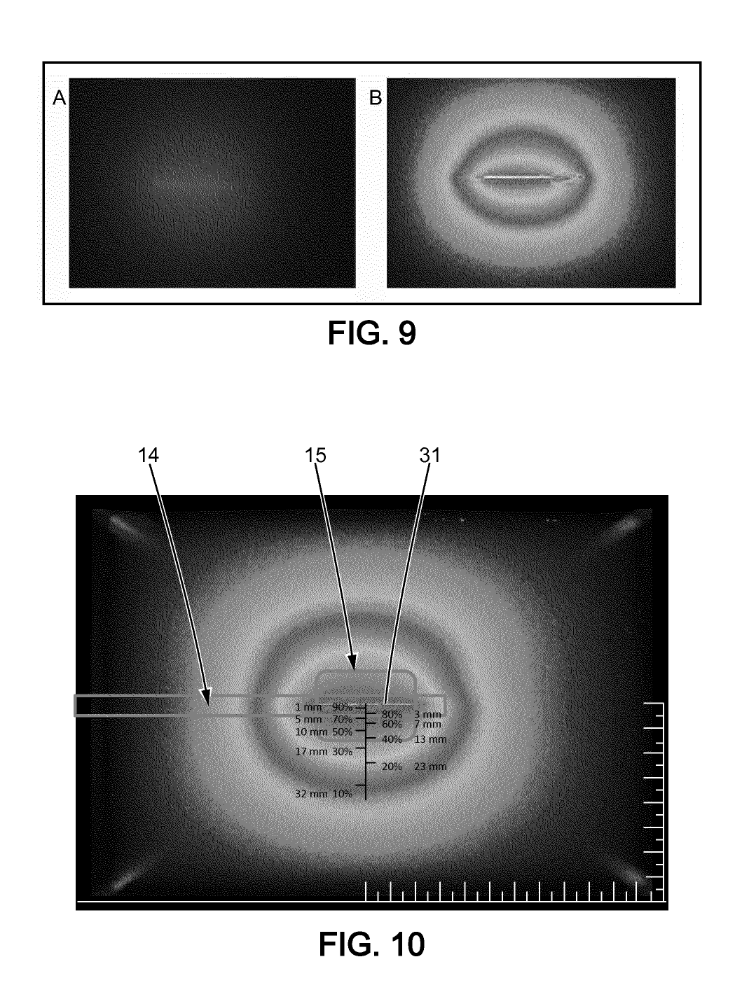

FIG. 9 illustrates A) an original picture of the illumination profile of the light emitting surface taken by a photography, B) a computed picture of the illumination profile of the light emitting surface according to pixel intensity,

FIG. 10 is a representation of the illumination profile of the light emitting surface of the illuminating member of FIG. 2 showing fractions of light power as a function of distance to the light emitting surface,

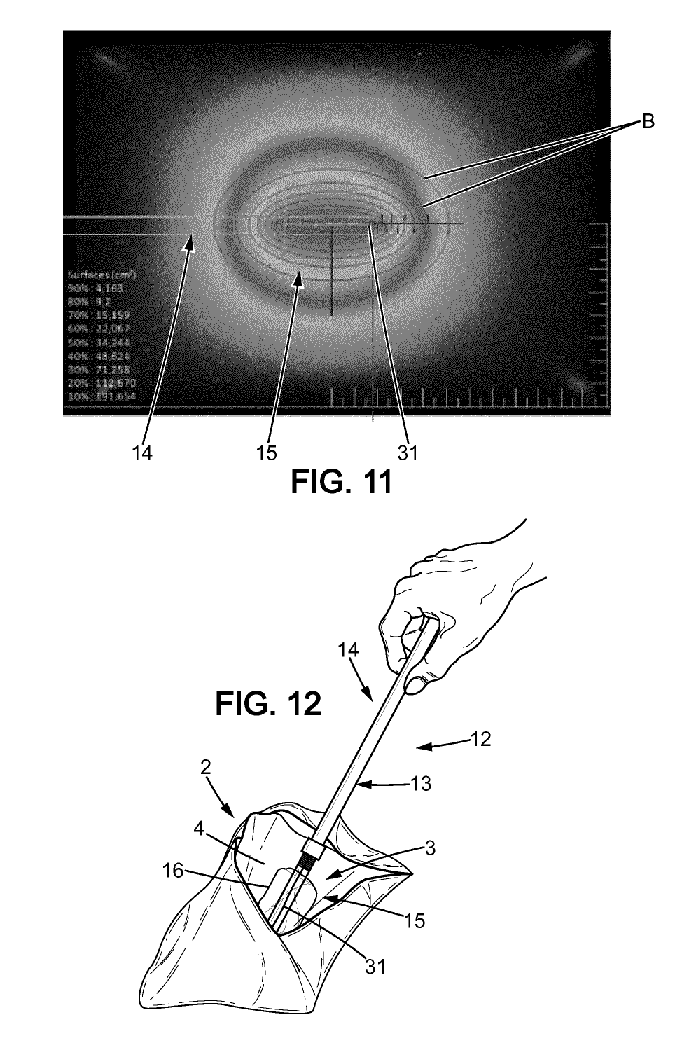

FIG. 11 is a representation of the illumination profile of the light emitting surface of the illuminating member of FIG. 2 showing respective illuminated areas of the fractions of light power,

FIG. 12 is a representation of an operator using the system for treatment of FIG. 1 for treating the internal surface of the pleural cavity of the body of a patient,

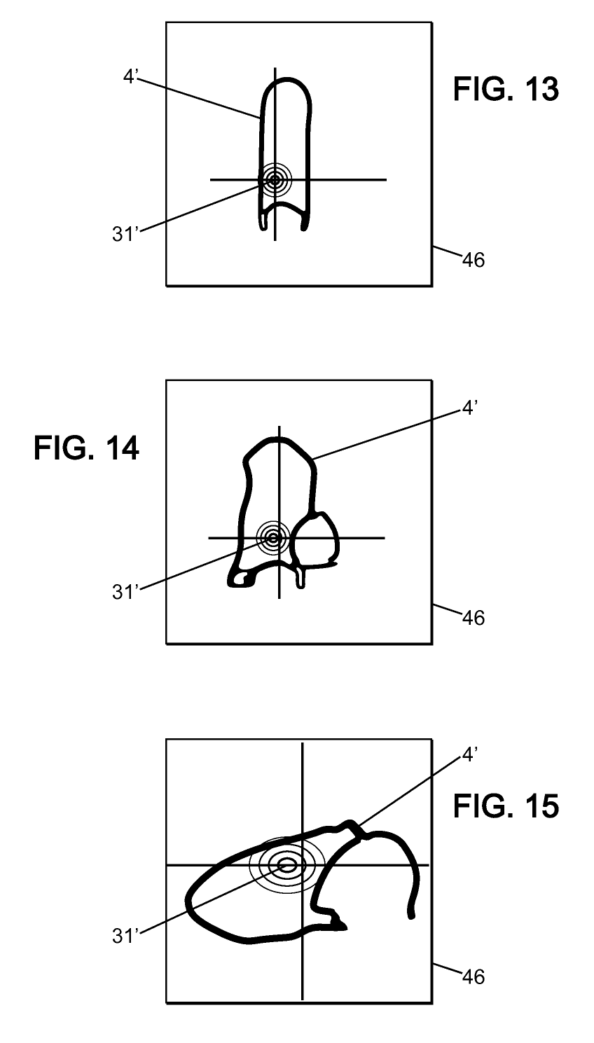

FIGS. 13 à 15 are representations of intra-operative dosimetry performed by the system for treatment of FIG. 1, wherein the position of the light emitting surface and the dose of light energy delivered to the internal surface to be treated are displayed in real-time on an image of the internal surface to be treated.

On the Figures, the same reference numbers refer to the same or similar elements.

FIG. 1 illustrates a system for treatment 1 by photodynamic therapy of an internal surface 4 of a patient's body 2. The system for treatment 1 is especially applied to the treatment of cancerous tumours of the pleural cavity 3 (illustrated on FIG. 12), such as malignant pleural mesothelioma (MPM) or peritoneal carcinoma.

The photodynamic therapy (PDT) relies upon activation of a photosensitizer compound, previously injected within the body 2 of the patient and absorbed by cells, by a suitable light to destroy tumour cells in which the photosensitizer compound remains a longer time.

The system for treatment 1 comprises:

an illuminating device 10 including a light emitting surface 31 for illuminating the internal surface 4 to be treated with a light adapted to activate the photosensitizer compound,

a positioning system 40 adapted to position in real-time the light emitting surface 21 within a reference frame XYZ,

an electronic unit 45 connected to the positioning system 40 and adapted to monitor in real-time a dose of light energy delivered to the internal surface 4 based on the illumination profile and the position of the light emitting surface 31.

On FIG. 2, the illuminating device 10 comprises an illuminating member 12 adapted to be manipulated by an operator, human or robot. The illuminating member 12 extends along a central axis A between opposed proximal 12a and distal 12b ends. In the illustrated embodiment, the central axis A is straight between the proximal 12a and distal 12b ends to ease its manipulation, although it could present one or more curvatures depending on the application. The illuminating member 12 comprises a handling part 14 extending from the proximal end 12a of the illuminating member 12 and a light diffusing part 15 arranged at the distal end 12b of the illuminating member 12.

The illuminating member 12 comprises a sterile outer sheath 13, for example cylindrical of circular cross-section, centered on the central axis A. The sheath 13 presents has an overall rigidity over the handling part 14 to further ease manipulation of the illuminating member 12. At the light diffusing part 15 of the illuminating member 12, the sheath 13 comprises an enlarged portion 16, cylindrical of circular cross-section with a diameter greater than that at the handling part 14, centered on the central axis A. The enlarged portion 16 is filed with a light diffusing solution, such as a diffusing solution of intralipide at 0.01% (1 mL of intralipide at 20% within 2 L of water).

The illuminating member 12 also comprises a core 30 carrying the light emitting surface 31 and inserted in the sheath 13 along the central axis A so that the light emitting surface 31 is centered within the enlarged portion 16 of the sheath 13. The core 30 is an optical fiber having a proximal end 30a and a distal end 30b which carries the light emitting surface 31. In a particular embodiment, the light emitting surface 31 is arranged along a portion of a lateral surface extending around an axis of the optical fiber 30 so that light may be emitted transversely with respect to axis of the optical fiber 30 and, when mounted within the sheath 13, to the central axis A of the illuminating member 12.

The illuminating device 10 further comprises a light source 32 and, in particular a laser light source, connected to the proximal end 30a of the optical fiber 30 and adapted to emit the light at determined wavelength and power so as to activate the photosensitizer compound.

In an exemplary non-limitative embodiment, the sheath 13 comprises a sterile orotracheal intubation (TOT) probe 17 having a proximal portion 17a and a distal portion 17b which comprises an inflatable balloon forming the enlarged portion 16. The sheath 13 also comprises an hollow tub 7 inserted within the proximal portion of the orotracheal intubation (TOT) probe 17 so as to rigidify and remove, or at least reduce, the curvature of the TOT probe 17. The tub 18 together with the proximal portion 17a of the TOT probe 17 forms the handling part 14 of the illuminating member 12 whereas the distal portion 17b of the TOT probe 17 is part of the light diffusing part 15 of the illuminating member 12. For example, the tube 18 is made of carbon, 280 mm in length, 8 mm in external diameter and 7 mm in internal diameter, and the TOT probe 17 is a probe Rüschelit® Super safetyclear.

The sheath 13 further comprises a hollow end cap 19, for example made of Plexiglas and 68 mm in length, having an opened end 19a fixed to a distal end 18b of the tub 18, and an opposed closed end 19b. The end cap 19, which is part of the light diffusing part 15, is adapted to receive the distal end 30b of the optical fiber 30 so as to maintain the light emitting surface 31 along the central axis A and to prevent it from contacting an inner surface of the TOT probe 17. The end cap 19 has a notch 20 at the vicinity of its opened end 19a.

A sterile cap 21 of the sheath 13 is attached to an opened distal end of the distal portion 17b of the TOT probe 17 beyond the end cap 19. The cap 21 is, for example, made of PVC, 20 mm in length and 8.3 mm in diameter.

The optical fiber 30 as core, for example a diffusing cylindrical optical fiber Medlight® of 38 mm, is inserted within the tube 18 and the end cap 19 and then fixed to a proximal end of the proximal portion 17a of the TOT probe 17 by an appropriate lock 22, such as a Luer lock.

The proximal end 30a of the optical fiber 30 is connected to the laser light source 32 emitting the appropriate light. The laser light source may have a power of 3 W and emits a light at a wavelength of 635 nm, such as the medical laser Ceramoptec®, Dioden-laser 635+−3 nm CW 3 Watt.

The light emitting surface 31 of the optical fiber 30 emits the light with a distribution of light power comprising fractions of light power decreasing from a maximum at the light emitting surface 31.

In order to enable homogenous and complete illumination of the internal surface 4, the system for treatment 1 performs dosimetry, that is a follow-up of dose of light energy delivered to the internal surface 4. Dosimetry depends on the power of the laser, the surface to illuminate and the time of illumination.

To that end, an illumination profile that provides respective illuminated areas for a plurality of fractions of light power is determined.

A theoretical diffusion of the light emitting surface 31 has been modelled by measuring the emitted light power. Two complementary methods are used: an isotropic sensor coupled to a wattmeter enabling a punctual and accurate estimation of an efficient attenuation coefficient μ_{eff}, and a digital photography enabling generalization to the whole space. Indeed, after interaction with the diffusing solution, photons emitted by the light emitting surface 31 underwent absorption and diffusion phenomena, modelled by the efficient attenuation coefficient μ_{eff} of light. Theoretically, light power decreases in an exponential manner when moving away from the light source. In particular, the Beer-Lambert equation states that: I=I₀.exp(−μ_{eff}.z) wherein I is the intensity of light, I₀ is the incident intensity of light and z is the distance to the light emitting surface.

The efficient attenuation coefficient μ_{eff} can be calculated by:

plotting light power as a function of the distance to the light emitting surface 31,

obtaining by interpolation an equation of an exponential function thus plotted, the exponential function being of the form y=a.exp(−bx), and

assigning b to the efficient attenuation coefficient μ_{eff} by analogy with the Beer-Lambert equation.

The efficient attenuation coefficient μ_{eff} measured can then be used in the Beer-Lambert equation to calculate fractions of light power at given distances (in mm) from the light emitting surface 31, thereby defining the theoretical diffusion of the light.

To that end, dosimetry experimental measurements are performed on a test bench shown on FIG. 4.

In order to simulate intra-operative illumination of the internal surface 4 to be treated, the illuminating member 12 is attached to a tank 35 filled with a diffusing solution so that its light emitting surface 31 is arranged within the tank 35.

As shown on FIG. 5, light power can then be measured at different distances from the light emitting surface 31, with one appropriate sensor moved about the light emitting surface 31 or several appropriate sensors positioned about the light emitting surface 31. In the exemplary non-limitative embodiment, the tank 35 has a capacity of 2280 ml and is filled with a diffusing solution of intralipide at 0.01% (1 mL of intralipide at 20% within 2 L of water). A horizontal arm 37 extending above an opened top 36 of the tank 35 holds an isotropic sensor 39, such as the isotropic sensor Medlight® Model IP 85 (*3), placed in a tube 38 and connected to a Wattmeter, such as the Wattmeter Newport® 841-PE, precision of 0.1 nW. The isotropic sensor 39 is then moved in two perpendicular directions of a plane, and especially horizontally and vertically, about the light diffusing part 31 of the illuminating member 12. The isotropic sensor 39 is moved in a length L, for example of 95 mm, and in a height H, for example of 30 mm, with a pitch p, for example of 5 mm.

In order to judge the repeatability of the experimental measurements, these can be performed:

three times in standard conditions, that is with a first TOT probe 17, a first diffusing optical fiber 30 of 38 mm, a first isotropic sensor 39, a laser power of 1 W and a diffusing solution comprising water and intralipide at a concentration of 0.01%, and

by changing the experimental conditions and, for example, through:

implementation of two other TOT probes 17 (of the same type as the first one),

implementation of two other isotropic sensors 39 (of the same type as the first one),

implementation of two other diffusing optical fibers 30 of 38 mm (of the same type as the first one),

rotation of the TOT probe 17 of 0°, 90°, 180° and 270° so as to evaluate the repeatability of the measurements in a three-dimension space, variation of the laser power: 0.5 W and 3 W.

The experimental measurements in standard conditions are shown on the graph of FIG. 7. The light power decreases exponentially from the light emitting surface 31 of the optical fiber 30.

A comparison between measurements in standard conditions and other experimental conditions provides the following results:

regarding implementation of other TOT probes 17: average difference: 2.7 nW, maximum difference: 6.6 nW,

regarding implementation of other isotropic sensors 39: average difference: 4 nW, maximum difference: 8.3 nW,

regarding rotation of the TOT probe 17 of 0°, 90°, 180° and 270°: average difference: 2 nW, maximum difference: 9 nW,

regarding variation of the laser power: multiplying by 2 and 6 times the input power results in multiplying by 1.87 and 5.09, respectively, the output light power; a loss between input and output can be noted, the loss increasing as the multiplying factor increases.

The deviations are therefore low so that the experimental protocol can be regarded as repeatable whichever the isotropic sensor 39 and the TOT probe 17 are, and in every direction of the illuminating member 12.

Light power as a function of distance to the light emitting surface is plotted on FIG. 8 based on the three series of experimental measurements in standard conditions. The efficient attenuation coefficient μ_{eff} is obtained thanks to the equation of the interpolation exponential function y=a.exp(−bx), by assigning b to the efficient attenuation coefficient μ_{eff} in accordance with the Beer-Lambert equation.

In particular, the exponential function is y=87.394.exp(−0.063x) so that the efficient attenuation coefficient μ_{eff} is 0.63 cm⁻¹ (0.063 mm⁻¹).

In table 1 below, the efficient attenuation coefficient μ_{eff} is then used in the Beer-Lambert equation I=I₀.exp(−μ_{eff}.z) to calculate fractions of light power as a function of the distance to the light emitting surface 31, within the diffusing solution.

TABLE 1

| Distance to the light | Fraction of |
|---|---|
| emitting surface (mm) | light power |
| 0 | 100% |
| 0.5 | 72.98% |
| 1 | 53.26% |
| 1.5 | 38.87% |
| 2 | 28.37% |
| 2.5 | 20.70% |
| 3 | 15.11% |
| 3.5 | 11.03% |
| 4 | 8.05% |
| 4.5 | 5.87% |
| 5 | 4.29% |
| 5.5 | 3.13% |
| 6 | 2.28% |
| 6.5 | 1.67% |
| 7 | 1.22% |

For validating the efficient attenuation coefficient μ_{eff}, new experimental measurements about the light diffusing part 15 of the illuminating member 12 are made so as to compare new values with those obtained by the theoretical model at a given distance from the light emitting surface 31.

For example, as shown on FIG. 6, the light power can be measured along different directions, such as three parallel directions extending radially with respect to the central axis A (X coordinate 15, 20 and 25 respectively), with a determined pitch along a determined distance, such as each millimetre along 25 mm. The measurements can be repeated, for example in three series using the same standard conditions as previously defined except for a power of 3 W since the efficient attenuation coefficient μ_{eff} depends on the diffusing solution rather than on the delivered power.

Experimental (measured) and theoretical (calculated) power values have been compared through the error calculus: Error=(measured value−calculated value)/measured value.

An error of less than 15% can be considered as acceptable.

The error calculus is performed for each direction (X coordinate 15, 20 and 25 respectively) and for each series. Results are shown in table 2 below which provides the error percentage between the experimental values and the calculated values from the theoretical model for μ_{eff}=0.63 cm⁻¹.

TABLE 2

| X coordinate | Series 1 | Series 2 | Series 3 |
|---|---|---|---|
| 15 | 7.70% | 5.53% | 3.92% |
| 20 | 11.75% | 11.41% | 5.08% |
| 25 | 19.36% | 14.28% | 9.76% |

The average difference between the calculated values and the measured values is 9.87%.

The efficient attenuation coefficient μ_{eff} can then be adjusted and optimized by computer in order to minimize the error.

The optimum value of the efficient attenuation coefficient μ_{eff} is 0.705 cm⁻¹. Table 3 below provides the error percentage between the experimental values and the calculated values from the theoretical model for μ_{Jeff=}0.705 cm⁻¹.

TABLE 3

| X coordinate | Series 1 | Series 2 | Series 3 |
|---|---|---|---|
| 15 | 3.94% | 5.50% | 5.92% |
| 20 | 4.77% | 4.73% | 5.73% |
| 25 | 8.54% | 4.59% | 4.13% |

The average difference between the calculated values and the measured values is reduced to 5.32%.

Values of light power measured by the Wattmeter are considered as right and accurate. However, they only provide discrete information rather than continuous information as to the distribution of light power, so that no global spatial representation of the distribution of light power could be obtained.

To overcome this deficiency, respective illuminated areas for a plurality of the fractions of light power may be measured by taking a picture illustrating the distribution of light power, identifying the plurality of the fractions of light power and delimiting respective borders of the plurality of the fractions of light power, calculating an area of each border corresponding to one of the illuminated areas.

In particular, pixel intensity of a digital picture of the light emitting surface 31 arranged in the above disclosed test bench is measured.

A photography apparatus with a focal of 19 cm is arranged above the opened top 36 of the tank 35 to take a picture, shown on FIG. 9A, in standard conditions as previously defined except for the presence of the isotropic sensor.

As shown on FIGS. 9B and 10, the picture can be displayed with an image parameter gradient, such as a colour or contrast gradient, as a function of light intensity of pixels through an appropriate software. For example, 10 fractions of light power each corresponding to a tenth of the maximum light power are represented with the corresponding image parameters differing from each other.

The theoretical illumination profile is established based on the efficient attenuation coefficient μ_{eff} of 0.705 cm⁻¹ and the representation of the spatial diffusion derived from the picture shown on FIG. 11. It shows ellipsoidal borders B, the areas of which can be calculated based on measured semi-principal axes.

Areas of the borders B on which the fractions of light power are distributed can be measured as well as irradiance, which corresponds to the dose of energy administered to a surface within a given time period in W/cm². Table 4 below provides the illuminated areas and the irradiance for μ_{eff}=0.705 cm⁻¹ and a laser power of 3 W.

TABLE 4

| Fraction of | Surface | Irradiance |
|---|---|---|
| light power | (cm²) | (W/cm²) |
| 100% | 1.194 | 2.513 |
| 90% | 4.163 | 0.649 |
| 80% | 9.200 | 0.261 |
| 70% | 15.159 | 0.139 |
| 60% | 22.067 | 0.082 |
| 50% | 34.244 | 0.044 |
| 40% | 48.624 | 0.025 |
| 30% | 71.258 | 0.013 |
| 20% | 112.670 | 0.005 |
| 10% | 191.654 | 0.002 |

In order to optimize dosimetry, the positioning system 40 enable the position of the light emitting surface 31 of the illuminating member 12 to be monitored and followed-up in real-time.

The positioning system 40 comprises one or several positioning sensors 41 attached to the illuminating member 12 at known locations from the light emitting surface 31 and connected to the electronic unit 45. The positioning sensor 41 can then emit a signal representative of its position within the reference frame XYZ.

Although not limited thereto, the positioning system 40 can be an electromagnetic spatial tracking system comprising an electromagnetic sensor as positioning sensor 41, and a transmitter 42 adapted to generate an electromagnetic field. The transmitter 42 and the electromagnetic sensor 41 are connected to the electronic unit 45 so that the position of the electromagnetic sensor 41 may be detected within a given space covered by the electromagnetic field.

The electromagnetic sensor 41 is fixed to the illuminating member 12, for example in the notch 20 of the end cap 19. In the exemplary non-limitative embodiment, the electromagnetic sensor 41 is an electromagnetic sensor 6 DOF Model 180 trakSTAR®, the transmitter 42 is a transmitter Mid Range trakSTAR® emitting an electromagnetic field within a radius of 46 cm and the electronic unit 45 comprises a control unit 3D Guidance trakSTAR to which the electromagnetic sensor 41 and the transmitter 42 are connected. In this particular embodiment, although not limited thereto, the electromagnetic sensor 41 is arranged 26 mm above and 3 mm aside a middle of the light emitting surface 31.

The electronic unit 45 comprises a memory loaded with an image 4′ of the internal surface to be treated, preferably in three dimensions, acquired in an appropriate modality such as tomography or magneto resonance imaging. The image 4′ of the internal surface to be treated may be displayed on a displaying device 46 of the electronic unit 45 together with an image 31′ of the light emitting surface moving in-real time in accordance with its position detected by the electromagnetic spatial tracking system 40.

In order to put in correspondence the image 4′ of the internal surface in the reference frame XYZ of the electromagnetic spatial tracking system 40, appropriate markers are placed on the body 2 of the patient prior to the acquisition of the image 4′ of the internal surface to be treated. The markers are chosen so as to be visible by contrast on the image 4′ according to the modality. For example, in the case of a tomography image, balls made of paraffin and 5 mm in diameter, can be placed on the thorax of the patient 2 to appear in hyper-density on the image. The operator then has to touch the markers with the illuminating member 12 to enable the positioning system 40 to identify their coordinates and the electronic unit 45 to calculate a spatial transformation to arrange the image 4′ within the reference frame XYZ.

The illumination profile of the light emitting surface 31 may also be loaded in the memory of the electronic unit 45 so that the cumulated dose of light energy delivered to the internal surface 4 to be treated may be displayed in real-time. An image parameter, such as colour or contrast, may have a plurality of values each representative of a value of the dose of light energy. The electronic unit 45 may change in real-time the value of the image parameter in accordance with the value of the dose of light energy. These provisions ease the illumination of the internal surface 4 by the operator, the parts of the internal surface 4 that have been illuminated and the dose of light energy, deriving from duration during which the part has been exposed to a determined light power function of the distance to the light emitting surface 31, being identified on the displaying device 46.

In relation with FIGS. 12 to 15, the above disclosed system for treatment 1 may be implemented in a method for treatment by photodynamic therapy of an internal surface 4 of a patient's body 2.

Such method comprises the steps of injecting the photosensitizer compound, such as hematoporphyrin (Photofrin®) and temoporfin (Foscan), within the body 2 of the patient and of acquiring an image 4′ of the internal surface 4 to be treated in the appropriate modality.

The method also comprises preparation of the treatment by providing the illuminating device 10, the positioning system 40 and the electronic unit 45. Such preparation also includes determination of the illumination profile of the light emitting surface 31 as disclosed previously.

The illumination profile and the image 4′ of the internal surface are loaded in the electronic unit 45, as well as any other useful data, such as the desired dose of light energy and the laser power. The image 4′ of the internal surface to be treated may then be displayed on the displaying device 46 of the electronic unit 45 and matched with the reference frame XYZ of the positioning system 40 by pointing the markers on the body 2 of the patient.

After resection surgery and after a sufficient time has elapsed so that the photosensitizer compound remains only within tumour cells, the operator may move the light diffusing part 15 of the illuminating member 12 within the cavity 3 to be treated and approach this light diffusing part 15 to the internal surface 4 to be treated so as to activate the photosensitizer compound. The image 31′ of the light emitting surface is displayed in real-time on the image 4′ of the internal surface to be treated together with the dose of light energy delivered to the internal surface 4 to be treated. In particular, the image parameter representative of the dose of light energy, such as colour or contrast, may be changed in real-time on the image 4′ of the internal surface to be treated in accordance with the evolution of the value of the delivered dose of light energy.

With an appropriate updating rate of the image 4′, the doses of light energy are cumulated and fluence, which corresponds to the delivered dose of light energy par area unit in J/cm² and which is equal to the irradiance multiplied by the time of illumination, is updated.

As the dose of light energy is monitored, and displayed in the disclosed embodiment, in real-time, an indication of the areas not yet or not sufficiently treated is provided to the operator. The operator is thus guided to perform homogeneous and complete illumination of the cavity 3 until a determined threshold of illumination corresponding to a appropriate delivered dose of light energy, such as 60 J/cm², has been reached on the whole internal surface 4 or at least on most of the internal surface 4. The electronic unit 45 may be further adapted to stop illumination of the internal surface to be treated when the determined threshold of illumination has been reached. For example, the electronic unit may switch off the light source or instruct the operator to remove the illumination member from the cavity, especially in the case of a robotic operator, when the appropriate dose of light energy, such as 60 J/cm², has been delivered to the whole internal surface 4 or at least on most of the internal surface 4, such as on more than 75% of the internal surface 4, preferably on more than 90% of the internal surface 4.

1. System for treatment by photodynamic therapy of an internal surface of a patient's body, the internal surface being delimited by tissues comprising cells having a photosensitizer compound absorbed therein, the system for treatment comprising:
an illuminating device intended for illuminating the internal surface to be treated, the illuminating device including a light emitting surface for emitting a light adapted to activate the photosensitizer compound, the illuminating device being adapted to diffuse the light emitted by the light emitting surface with a distribution of light power comprising fractions of light power decreasing from a maximum at the light emitting surface;
an electronic unit adapted to monitor in real-time a dose of light energy delivered to the internal surface,
wherein the system for treatment further comprises a positioning system adapted to position in real-time the light emitting surface within a reference frame, and in that the light diffused by the light emitting surface has a determined illumination profile that provides respective illuminated areas for a plurality of the fractions of light power so that the dose of light energy delivered at each distance from the light emitting surface of the plurality of the fractions of light power is known, the electronic unit being connected to the positioning system and adapted to monitor in real-time the dose of light energy based on the illumination profile and the position of the light emitting surface.

2. System for treatment according to claim 1, wherein the illuminating device comprises an illuminating member extending along a central axis between opposed proximal and distal ends, the light emitting surface being arranged at the distal end and extending along the central axis so as to emit the light transversely with respect to the central axis (A).

3. System for treatment according to claim 2, wherein the illuminating member comprises:
a sheath having an enlarged portion arranged at the distal end, centered on the central axis and filed with a light diffusing solution; and
a core extending in the sheath and carrying the light emitting surface arranged within the enlarged portion.

4. System for treatment according to claim 3, wherein the core of the illuminating member is an optical fiber having a proximal end and a distal end which carries the light emitting surface, and wherein the illuminating device further comprises a laser light source connected to the proximal end of the optical fiber.

5. System for treatment according to claim 2, wherein the distribution of light power emitted by the light emitting surface has a rotational symmetry, the illuminated areas for the plurality of the fractions of light power being centered on the light emitting surface.

6. System for treatment according to claim 5, wherein the light emitting surface is unique and cylindrical along the central axis.

7. System for treatment according to claim 1, wherein the positioning system comprises at least one positioning sensor attached to the illuminating device and connected to the electronic unit, the positioning sensor being adapted to emit a signal representative of the position of said positioning sensor within the reference frame.

8. System for treatment according to claim 7, wherein the positioning system is an electromagnetic spatial tracking system comprising an electromagnetic sensor as positioning sensor, and a transmitter adapted to generate an electromagnetic field.

9. System for treatment according to claim 1, wherein the electronic unit is provided with an image of the internal surface to be treated and comprises a displaying device adapted to display the image of the internal surface to be treated, the electronic unit being adapted to display in real-time an image of the light emitting surface on the image of the internal surface to be treated.

10. System for treatment according to claim 9, wherein the electronic unit is further adapted to display in real-time the dose of light energy delivered to the internal surface to be treated.

11. System for treatment according to claim 10, wherein the displaying device comprises an image parameter representative of the dose of light energy on the image of the internal surface to be treated, the image parameter comprising a plurality of values each representative of a value of the dose of light energy, the electronic unit being adapted to change in real-time the value of the image parameter in accordance with the value of the dose of light energy.

12. System for treatment according to claim 1, wherein the electronic unit is further adapted to stop illumination of the internal surface to be treated when a determined threshold of illumination has been reached.

13. Method for preparation of a system for treatment by photodynamic therapy of an internal surface of a patient's body, the internal surface being delimited by tissues comprising cells having a photosensitizer compound absorbed therein, the method for preparation comprising the steps of:
providing an illuminating device intended for illuminating the internal surface to be treated, the illuminating device including a light emitting surface for emitting a light adapted to activate the photosensitizer compound, the illuminating device being adapted to diffuse the light emitted by the light emitting surface with a distribution of light power comprising fractions of light power decreasing from a maximum at the light emitting surface;
determining an illumination profile of the light diffused by the light emitting surface, the illumination profile providing respective illuminated areas for a plurality of the fractions of light power;
providing a positioning system adapted to position in real-time the light emitting surface within a reference frame;
providing an electronic unit connected to the positioning system and adapted to monitor in real-time a delivered dose of light energy based on the illumination profile and the position of the light emitting surface.

14. Method for preparation according to claim 13, wherein the step of determining the illumination profile comprises:
measuring an efficient attenuation coefficient μ_{eff} of light,
calculating the fractions of light power based on the Beer-Lambert equation: I=I₀.exp(−μ_{eff}.z) wherein I is the intensity of light, I₀ is the incident intensity of light and z is the distance to the light emitting surface; and,
measuring the respective illuminated areas for the plurality of the fractions of light power.

15. Method for preparation according to claim 14, wherein measuring the efficient attenuation coefficient comprises measuring light power at a plurality of distances from the light emitting surface, plotting an interpolation curve y=a.exp(−bx) representing light power as a function of a distance to the light emitting surface, and assigning b to the efficient attenuation coefficient μ_{eff} in accordance with the Beer-Lambert equation.

16. Method for preparation according to claim 14, wherein measuring the respective illuminated areas for the plurality of the fractions of light power comprises taking a picture illustrating the distribution of light power, identifying the plurality of the fractions of light power and delimiting respective borders of the plurality of the fractions of light power, calculating an area of each border corresponding to one of the illuminated areas.

17. Method for preparation according to claim 13, further comprising the steps of:
acquiring an image of the internal surface to be treated;
providing the electronic unit with the image of the internal surface to be treated;
displaying the image of the internal surface to be treated on a displaying device of the electronic unit.

18. Method for preparation according to claim 17, further comprising the step of arranging the image of the internal surface to be treated in the reference frame of the positioning system.
